SEQUENCE LISTING (24 sequences)

SEQ ID NO: 1 - DNA, 288 bp, Pseudoplectania nigrella
  CDS (1)..(285)
  sig_peptide (1)..(69)
  mat_peptide (166)..(285)
atgcaatttaccaccatcctctccatcggtatcaccgtcttcggacttctcaacaccggagcctttgcagcaccccagcctgttcccgaggcttacgctgtttctgatcccgaggctcatcctgacgattttgctggtatggatgcgaaccaacttcagaaacgtggatttggatgcaatggtccttgggatgaggatgatatgcagtgccacaatcactgcaagtctattaagggttacaagggaggttattgtgctaaggggggctttgtttgcaagtgttactag

SEQ ID NO: 2 - protein, 95 aa, Pseudoplectania nigrella
MQFTTILSIGITVFGLLNTGAFAAPQPVPEAYAVSDPEAHPDDFAGMDANQLQKRGFGCNGPWDEDDMQCHNHCKSIKGYKGGYCAKGGFVCKCY

SEQ ID NO: 3 - DNA, 273 bp, Eurotium amstelodami
  CDS (1)..(270)
  sig_peptide (1)..(60)
  mat_peptide (145)..(270)
atgcacttcaccaaggtctccaccattctttttaccatcttcgccgccggcatcatggctgctcccaccgaaggagtccgtgaggaagccgcccctggccaggaggtttaccccgacgaacctcctgcttctctgaccaagcgtggcttcggatgtcctggtgatgcctaccagtgcagtgaacactgcagggccctgggcggtggacgcactggaggatactgtgctggaccttggtatttgggtcaccctacctgcacctgttctttctaa

SEQ ID NO: 4 - protein, 90 aa, Eurotium amstelodami
MHFTKVSTILFTIFAAGIMAAPTEGVREEAAPGQEVYPDEPPASLTKRGFGCPGDAYQCSEHCRALGGGRTGGYCAGPWYLGHPTCTCSF

SEQ ID NO: 5 - DNA, 273 bp, Picea glauca
  CDS (1)..(270)
  sig_peptide (1)..(60)
  mat_peptide (151)..(270)
atgaagttcaccatctccatcatcgccgctctcgctttcttcgcccagggaatcgtggcagctcctgcgcctatccccgaggccgccgcggtggctgccccagaggccgagccaaaggcgttagatgagcttccggagttgcaaaagcgtggctttgggtgcaacggttggcctttcgaggacgatgagcagtgccataatcactgcaagaccattcctggttataagggtggctactgtgccaacgttggcaccacttgcaagtgctactag

SEQ ID NO: 6 - protein, 90 aa, Picea glauca
MKFTISIIAALAFFAQGIVAAPAPIPEAAAVAAPEAEPKALDELPELQKRGFGCNGWPFEDDEQCHNHCKTIPGYKGGYCANVGTTCKCY

SEQ ID NO: 7 - DNA, 189 bp, Crassostrea virginica
  CDS (1)..(186)
  sig_peptide (1)..(66)
  mat_peptide (67)..(186)
atgaaagtgtttgtacttcttacaatagcagtaatgcttatggtatctgccgacgttgctttggccggctttggctgtcctttgaaccggtaccagtgtcactcacattgccaatccattggtcgtaaaggtggatactgtggtgggtggtggagttttacatgcacatgctaccgcactaaaaagtag

SEQ ID NO: 8 - protein, 62 aa, Crassostrea virginica
MKVFVLLTIAVMLMVSADVALAGFGCPLNRYQCHSHCQSIGRKGGYCGGWWSFTCTCYRTKK

SEQ ID NO: 9 - DNA, 189 bp, Mesobuthus gibbosus
  CDS (1)..(186)
  sig_peptide (1)..(72)
  mat_peptide (73)..(186)
atgaaaaccattgtacttcttttcgtgttggctttagtattctgcactcttgaaatgggaatggtggaagccggattcggttgtccattcaatcaaggaagatgtcacagacattgtcgaagtattcgtcgaagaggaggatattgcgatggatttttgaaacaaagatgtgtttgttatcggagataa

SEQ ID NO: 10 - protein, 62 aa, Mesobuthus gibbosus
MKTIVLLFVLALVFCTLEMGMVEAGFGCPFNQGRCHRHCRSIRRRGGYCDGFLKQRCVCYRR

SEQ ID NO: 11 - DNA, 198 bp, Crassostrea gigas
  CDS (1)..(195)
  sig_peptide (1)..(66)
  mat_peptide (67)..(195)
atgaaagtattcgttcttttaacactagctgtccttctgatggtttctgcagacatggcttttgctggatttgggtgtccgggtaaccagttaaagtgcaacaatcactgcaagtccattagttgtagagcgggctactgtgatgcagccacgctctggttaagatgtacatgtaccgattgtaatggaaagaagtaa

SEQ ID NO: 12 - protein, 65 aa, Crassostrea gigas
MKVFVLLTLAVLLMVSADMAFAGFGCPGNQLKCNNHCKSISCRAGYCDAATLWLRCTCTDCNGKK

SEQ ID NO: 13 - DNA, 195 bp, Crassostrea virginica
  CDS (1)..(192)
  sig_peptide (1)..(66)
  mat_peptide (67)..(192)
atgaaagtgtttgttcttctaacaatagctgtcatgcttttggtatctgccgatgtagctactgcagataacggatgtccccgtcgtccgagaatctgtcacaatcggtgcatatacaaaggtcgtagaggcggaaaatgtgtcggaaagtggagaagcttatgcgaatgcatctacccatcgaaggccgggtga

SEQ ID NO: 14 - protein, 64 aa, Crassostrea virginica
MKVFVLLTIAVMLLVSADVATADNGCPRRPRICHNRCIYKGRRGGKCVGKWRSLCECIYPSKAG

SEQ ID NO: 15 - DNA, 207 bp, Aspergillus oryzae
  CDS (1)..(207)
  sig_peptide (1)..(54)
  mat_peptide (79)..(207)
atgaaacttctgacggtcgccttttcccttcttcttctcgggcaagtccatgccagtcctttggtactcgacaaaaggtcttcctgccagttgggtgacgtctgggacctcaatgctgcagacgccgcctgcagcgcttcgtgtgccattcaacacggcgacaaacacggcggacactgcgataagaacaaggtctgcgtctgcaat

SEQ ID NO: 16 - protein, 69 aa, Aspergillus oryzae
MKLLTVAFSLLLLGQVHASPLVLDKRSSCQLGDVWDLNAADAACSASCAIQHGDKHGGHCDKNKVCVCN

SEQ ID NO: 17 - DNA, 362 bp, Artificial
  Pleactasin encoding sequence with intron (see Example 1)
ggatccaccatgcaatttaccaccatcctctccatcggtatcaccgtcttcggacttctcaacaccggagcctttgcagcaccccagccggtacccgaggcttacgctgtttctgatcccgaggctcatcctgacgattttgctggtatggatgcgaaccaacttcagaaacgtggatttggatgcaatggtccttgggatgaggatgatatgcagtgccacaagtaagaatcacttataactagtagattaagccaagagtattggaactgatgataaatagtcactgcaagtctattaagggttacaagggaggttattgtgctaaggggggctttgtttgcaagtgttactagctcgag

SEQ ID NO: 18 - DNA, 303 bp, Artificial
  Intron-less MluN1 containing sequence encoding Plectasin (see Example 3)
ggatccaccatgcaatttaccaccatcctctccatcggtatcaccgtcttcggactggccaacaccggagcctttgcagcaccccagccggtacccgaggcttacgctgtttctgatcccgaggctcatcctgacgattttgctggtatggatgcgaaccaacttcagaaacgtggatttggatgcaatggtccttgggatgaggatgatatgcagtgccacaatcactgcaagtctattaagggttacaagggaggttattgtgctaaggggggctttgtttgcaagtgttactagctcgag

SEQ ID NO: 19 - DNA, 358 bp, Artificial
  Plectasin encoding sequence with intron of pMT2899 (see Example 3)
ggatccaccatgcaatttaccaccatcctctccatcggtatcaccgtcttcggactgggtatgtacaccacccccttgcgtctgatctgtgacatatgtagctgactggtcagccaacaccggagcctttgcagcaccccagccggtacccgaggcttacgctgtttctgatcccgaggctcatcctgacgattttgctggtatggatgcgaaccaacttcagaaacgtggatttggatgcaatggtccttgggatgaggatgatatgcagtgccacaatcactgcaagtctattaagggttacaagggaggttattgtgctaaggggggctttgtttgcaagtgttactagctcgag

SEQ ID NO: 20 - DNA, 362 bp, Artificial
  Plectasin encoding sequence of pMT2900 (see Example 3)
ggatccaccatgcaatttaccaccatcctctccatcggtatcaccgtcttcggactgggtaagaatcacttataactagtagattaagccaagagtattggaactgatgataaacagccaacaccggagcctttgcagcaccccagccggtacccgaggcttacgctgtttctgatcccgaggctcatcctgacgattttgctggtatggatgcgaaccaacttcagaaacgtggatttggatgcaatggtccttgggatgaggatgatatgcagtgccacaatcactgcaagtctattaagggttacaagggaggttattgtgctaaggggggctttgtttgcaagtgttactagctcgag

SEQ ID NO: 21 - DNA, 34 bp, Artificial
  Primer A (see Example 5)
tcttggatccaccatgcacttcaccaaggtctcc

SEQ ID NO: 22 - DNA, 33 bp, Artificial
  Primer B (see Example 5)
tcttctcgagttagaaagaacaggtgcaggtac

SEQ ID NO: 23 - DNA, 386 bp, Eurotium amstelodami
  CDS (10)..(195)
  sig_peptide (10)..(69)
  mat_peptide (154)..(377)
  Intron (196)..(240)
  CDS (241)..(305)
  Intron (306)..(358)
  CDS (359)..(377)
ggatccaccatgcacttcaccaaggtctccaccattctttttaccatcttcgccgccggcatcatggctgctcccaccgaaggagtccgtgaggaagccgcccctggccaggaggtttaccccgacgaacctcctgcttctctgaccaagcgtggcttcggatgtcctggtgatgcctaccagtgcagtgaacacgtatgtcctgctgctatctagagtgaatgaagctaatgaatatagtgcagggccctgggcggtggacgcactggaggatactgtgctggaccttggtatttgggtcacccgtatgttgcctagatatttaattgtttatagtcctttactgatgagcttatagtacctgcacctgttctttctaactcgag

SEQ ID NO: 24 - protein, 90 aa, Eurotium amstelodami; identical to SEQ ID NO: 4

(19) United States
(12) Patent Application Publication
(10) Pub. No.: US 2006/0211089 A1
(43) Pub. Date: Sep. 21, 2006

(54) Expression of defensins in filamentous fungi

(75) Inventors: Hans-Henrik Kristensen Hoegenhaug, Holte (DK); Kirk Matthew Schnorr, Holte (DK); Mogens Trier Hansen, Lynge (DK)

Correspondence Address: NOVOZYMES NORTH AMERICA, INC., 500 FIFTH AVENUE, SUITE 1600, NEW YORK, NY, 10110, US

(73) Assignee: Novozymes A/S, Bagsvaerd (DK)

(21) Appl. No.: 11/377,883

(22) Filed: Mar. 15, 2006

Related U.S. Application Data
(60) Provisional application No. 60/662,538, filed on Mar. 16, 2005.

(30) Foreign Application Priority Data
Mar. 16, 2005 (DK) 2005 00375

(51) Int. Cl. C07K 14/435; C07H 21/04; C12P 21/06; C12N 15/74; C12N 1/16; C07K 14/415

(52) U.S. Cl. 435/69.1; 435/484; 435/254.3; 530/350; 536/23.5

(57) ABSTRACT

The present invention relates to recombinant expression of defensin antimicrobial peptides in fermentation of filamentous fungi.

### CROSS-REFERENCE TO RELATED APPLICATIONS

This application claims priority or the benefit under 35 U.S.C. 119 of Danish application no. PA 2005 00375 filed Mar. 16, 2005 and U.S. provisional application No. 60/662,538 filed Mar. 16, 2005, the contents of which are fully incorporated herein by reference.

### Field of the Invention

The present invention relates to recombinant expression of defensin antimicrobial peptides in filamentous fungi.

### BACKGROUND OF THE INVENTION

Defensins belong to a class of small antimicrobial peptides. They are capable of killing a broad spectrum of microorganisms, some of which are becoming increasingly resistant towards traditional antibiotics. For that reason it is also becoming more and more interesting to be capable of producing defensins in large amounts at a low cost.

Since defensins usually only comprise 30-50 amino acid residues, they are often difficult to produce efficiently by use of recombinant fermentation methods. Chemical peptide synthesis is an alternative method, but this is too expensive when peptides exceed 25-30 amino acid residues. Another complication is that defensins comprise a distinctive cysteine pattern, which is difficult to create by chemical synthesis.

Accordingly, it is an object of the present invention to provide methods for obtaining improved expression levels of defensin antimicrobial peptides in recombinant fermentation of filamentous fungi.

### SUMMARY OF THE INVENTION

The inventors of the present invention have found that by inserting one or more intron sequences in a nucleic acid construct, which directs expression of a defensin, the recombinant expression level may be improved by more than 50% compared to using a nucleic acid construct with no intron sequences. The intron sequence(s) may be inserted anywhere in the nucleic acid construct, such as in the mature defensin encoding sequence, or even in a signal peptide encoding sequence.

Accordingly, the present invention relates to a recombinant filamentous fungal host cell, comprising a nucleic acid construct, which nucleic acid construct comprises a foreign nucleic acid sequence encoding a defensin and one or more intron sequence(s).

In a second aspect, the invention relates to a method for recombinant production of a defensin in a filamentous fungal host cell, which includes cultivating the filamentous fungal host cell comprising a nucleic acid construct, which nucleic acid construct comprises a nucleic acid sequence encoding the defensin peptide and one or more intron sequence(s); and recovering the defensin peptide.

In a third aspect, the invention relates to use of a nucleic acid construct, which comprises a nucleic acid sequence encoding a defensin peptide and one or more intron sequence(s), for improving the recombinant expression level of the defensin in a filamentous fungal host cell.

Definitions

Antimicrobial activity: The term “antimicrobial activity” is defined herein as an activity which is capable of killing or inhibiting growth of microbial cells. In the context of the present invention the term “antimicrobial” is intended to mean that there is a bactericidal and/or a bacteriostatic and/or fungicidal and/or fungistatic effect and/or a virucidal effect, wherein the term “bactericidal” is to be understood as capable of killing bacterial cells. The term “bacteriostatic” is to be understood as capable of inhibiting bacterial growth, i.e., inhibiting growing bacterial cells. The term “fungicidal” is to be understood as capable of killing fungal cells. The term “fungistatic” is to be understood as capable of inhibiting fungal growth, i.e., inhibiting growing fungal cells. The term “virucidal” is to be understood as capable of inactivating virus. The term “microbial cells” denotes bacterial or fungal cells (including yeasts).

In the context of the present invention the term “inhibiting growth of microbial cells” is intended to mean that the cells are in the non-growing state, i.e., that they are not able to propagate.

For purposes of the present invention, antimicrobial activity may be determined according to the procedure described by Lehrer et a/., 1991, Journal of Immunological Methods, 137(2): 167-174. Alternatively, antimicrobial activity may be determined according to the NCCLS guidelines from CLSI (Clinical and Laboratory Standards Institute; formerly known as National Committee for Clinical and Laboratory Standards).

Defensins having antimicrobial activity may be capable of reducing the number of living cells of Escherichia coli (DSM 1576) to 1/100 after 8 hours (preferably after 4 hours, more preferably after 2 hours, most preferably after 1 hour, and in particular after 30 minutes) incubation at 20° C. in an aqueous solution of 25% (w/w); preferably in an aqueous solution of 10% (w/w); more preferably in an aqueous solution of 5% (w/w); even more preferably in an aqueous solution of 1% (w/w); most preferably in an aqueous solution of 0.5% (w/w); and in particular in an aqueous solution of 0.1% (w/w) of the defensins having antimicrobial activity.

Defensins having antimicrobial activity may also be capable of inhibiting the outgrowth of Escherichia coli (DSM 1576) for 24 hours at 25° C. in a microbial growth substrate, when added in a concentration of 1000 ppm; preferably when added in a concentration of 500 ppm; more preferably when added in a concentration of 250 ppm; even more preferably when added in a concentration of 100 ppm; most preferably when added in a concentration of 50 ppm; and in particular when added in a concentration of 25 ppm.

Defensins having antimicrobial activity may be capable of reducing the number of living cells of Bacillus subtilis (ATCC 6633) to 1/100 after 8 hours (preferably after 4 hours, more preferably after 2 hours, most preferably after 1 hour, and in particular after 30 minutes) incubation at 20° C. in an aqueous solution of 25% (w/w); preferably in an aqueous solution of 10% (w/w); more preferably in an aqueous solution of 5% (wlw); even more preferably in an aqueous solution of 1% (w/w); most preferably in an aqueous solution of 0.5% (w/w); and in particular in an aqueous solution of 0.1 % (w/w) of the defensins having antimicrobial activity.

Defensins having antimicrobial activity may also be capable of inhibiting the outgrowth of Bacillus subtilis (ATCC 6633) for 24 hours at 25° C. in a microbial growth substrate, when added in a concentration of 1000 ppm; preferably when added in a concentration of 500 ppm; more preferably when added in a concentration of 250 ppm; even more preferably when added in a concentration of 100 ppm; most preferably when added in a concentration of 50 ppm; and in particular when added in a concentration of 25 ppm.

The Defensins of the present invention have at least 20%, preferably at least 40%, more preferably at least 50%, more preferably at least 60%, more preferably at least 70%, more preferably at least 80%, even more preferably at least 90%, most preferably at least 95%, and even most preferably at least 100% of the antimicrobial activity of the defensin consisting of the amino acid sequence shown as amino acids 1 to 42 of SEQ ID NO: 2.

cDNA: The term “cDNA” is defined herein as a DNA molecule which can be prepared by reverse transcription from a mature, spliced, mRNA molecule obtained from a eukaryotic cell. cDNA lacks intron sequences that are usually present in the corresponding genomic DNA. The initial, primary RNA transcript is a precursor to mRNA which is processed through a series of steps before appearing as mature spliced mRNA. These steps include the removal of intron sequences by a process called splicing. cDNA derived from mRNA lacks, therefore, any intron sequences.

Nucleic acid construct: The term “nucleic acid construct” as used herein refers to a nucleic acid molecule, either single- or double-stranded, which is isolated from a naturally occurring gene or which is modified to contain segments of nucleic acids in a manner that would not otherwise exist in nature. The term nucleic acid construct is synonymous with the term “expression cassette” when the nucleic acid construct contains the control sequences required for expression of a coding sequence of the present invention.

Control sequence: The term “control sequences” is defined herein to include all components, which are necessary or advantageous for the expression of a polynucleotide encoding a defensin. Each control sequence may be native or foreign to the nucleotide sequence encoding the defensin. Such control sequences include, but are not limited to, a leader, polyadenylation sequence, propeptide sequence, promoter, signal peptide sequence, and transcription terminator. At a minimum, the control sequences include a promoter, and transcriptional and translational stop signals. The control sequences may be provided with linkers for the purpose of introducing specific restriction sites facilitating ligation of the control sequences with the coding region of the nucleotide sequence encoding a defensin.

Operably linked: The term “operably linked” denotes herein a configuration in which a control sequence is placed at an appropriate position relative to the coding sequence of the polynucleotide sequence such that the control sequence directs the expression of the coding sequence of a defensin.

Coding sequence: When used herein the term “coding sequence” means a nucleotide sequence, which directly specifies the amino acid sequence of its protein product. The boundaries of the coding sequence are generally determined by an open reading frame, which usually begins with the ATG start codon or alternative start codons such as GTG and TTG. The coding sequence may a DNA, cDNA, or recombinant nucleotide sequence.

Expression: The term “expression” includes any step involved in the production of the defensin including, but not limited to, transcription, post-transcriptional modification, translation, post-translational modification, and secretion.

Expression vector: The term “expression vector” is defined herein as a linear or circular DNA molecule that comprises a polynucleotide encoding a defensin peptide, and which is operably linked to additional nucleotides that provide for its expression.

Host cell: The term “host cell”, as used herein, includes any cell type which is susceptible to transformation, transfection, transduction, and the like with a nucleic acid construct of the present invention.

Modification: The term “modification” means herein any chemical modification of the defensin. The modification(s) can be substitution(s), deletion(s) and/or insertions(s) of the amino acid(s) as well as replacement(s) of amino acid side chain(s); or use of unnatural amino acids with similar characteristics in the amino acid sequence. In particular the modification(s) can be amidations, such as amidation of the C-terminus.

Identity: The relatedness between two amino acid sequences or between two nucleotide sequences is described by the parameter “identity”.

For purposes of the present invention, the degree of identity between two amino acid sequences is determined by using the program FASTA included in version 2.0x of the FASTA program package (see W. R. Pearson and D. J. Lipman, 1988, “Improved Tools for Biological Sequence Analysis”, PNAS 85: 2444-2448; and W. R. Pearson (1990) “Rapid and Sensitive Sequence Comparison with FASTP and FASTA”, Methods in Enzymology 183: 63-98). The scoring matrix used was BLOSUM50, gap penalty was −12, and gap extension penalty was −2.

The degree of identity between two nucleotide sequences is determined using the same algorithm and software package as described above. The scoring matrix used was the identity matrix, gap penalty was −16, and gap extension penalty was −4.

Alternatively, an alignment of two amino acid sequences is determined by using the Needle program from the EMBOSS package (http://emboss.org) version 2.8.0. The Needle program implements the global alignment algorithm described in Needleman and Wunsch, 1970, J. Mol. Biol. 48: 443-453. The substitution matrix used is BLOSUM62, gap opening penalty is 10, and gap extension penalty is 0.5.

The degree of identity between an amino acid sequence of the present invention (“invention sequence”; e.g., amino acids 1 to 40 of SEQ ID NO: 2) and a different amino acid sequence (“foreign sequence”) is calculated as the number of exact matches in the overlap of an alignment of the two sequences, divided by the length of the “invention sequence” or the length of the “foreign sequence”, whichever is the shortest. The result is expressed in percent identity.

An exact match occurs when the “invention sequence” and the “foreign sequence” have identical amino acid residues in the same positions of the overlap. The length of a sequence is the number of amino acid residues. in the sequence (e.g., the length of amino acids 1 to 40 of SEQ ID NO: 2 is 40).

### DETAILED DESCRIPTION

Defensins

The defensins of the invention is any antimicrobial peptide recognized by a person skilled in the art as belonging to the defensin class of antimicrobial peptides. To determine if an antimicrobial peptide is a defensin according to the invention, the amino acid sequence is preferably compared with the hidden markov model profiles (HMM profiles) of the well-known PFAM database (see Example 6).

The defensins may belong to the alpha-defensin class, the beta-defensin class, the theta-defensin class, the arthropod defensin class, the insect defensin class, the plant defensin class.

The defensins may also be synthetic defensins sharing the characteristic features of any of the defensin classes.

In an embodiment, the amino acid sequence of a defensin according to the invention comprises 4, 5, 6, 7, 8, 9, or 10 cysteine residues, preferably 6, 7, 8, 9, or 10 cysteine residues, more preferably 6, 8, or 10 cysteine residues, and most preferably 6 or 8 cysteine residues.

Examples of defensins include, but are not limited to, alpha-Defensin HNP-1 (human neutrophil peptide) HNP-2 and HNP-3; beta-Defensin-12, Drosomycin, Heliomicin, gamma1-purothionin, Insect defensin A, and the defensins disclosed in PCT applications WO 99/53053 (Rhone Poulenc Agrochimie) and WO 02/085934 (Entomed), which are hereby incorporated by reference; or those set forth in the mature amino acid sequences of SEQ ID NO: 2, SEQ ID NO: 4, SEQ ID NO: 6, SEQ ID NO: 8, SEQ ID NO: 10, SEQ ID NO: 12, SEQ ID NO: 14 and SEQ ID NO: 16 — or amino acid sequences which are at least 60%, preferably 70%, more preferably 80%, even more preferably 90%, and most preferably 95% identical to these sequences. A defensin according to the invention may furthermore comprise one or more chemical modifications compared to these amino acid sequences.

Alpha-defensins may be defined as antimicrobial peptides comprising the amino acid sequence:
C-X₁-C-X₂-C-X₃-C-X4-C-C
wherein

X₁ represents 1 amino acid; preferably X₁=Y, F, A, R, I, S, T, H or V; more preferably X₁=Y, F, A or R; even more preferably X₁=Y or F; most preferably X₁=Y;

X₂ represents 4 or 5 amino acids; preferably X₂ represents 4 amino acids; more preferably X₂=Z₁-Z₂-Z₃-Z₄, wherein

Z₁ represents any amino acid; preferably Z₁=R, T or K; more preferably Z₁=R;

Z₂ represents any amino acid; preferably Z₂=R, I, T, K;

Z₃ represents any amino acid; preferably Z₃=R, P or G;

Z₄ represents any amino acid; preferably Z₄=G, A or R;

X₃ represents 9 amino acids; preferably X₃=Z₁-Z₂-Z₃-Z₄-Z₅-Z₆-Z₇-G-Z₈, wherein

Z₁ represents any amino acid; preferably Z₁=K, L or R;

Z₂ represents any amino acid; preferably Z₂=R, F, A, S or G;

Z₃ represents any amino acid; preferably Z₃=R, G, P or T; more preferably Z₃=R or G;

Z₄ represents any amino acid; Z₄=E or Y; preferably Z₄=E;

Z₅ represents any amino acid; preferably Z₅=R, H or S;

Z₆ represents any amino acid; preferably Z₆=R, M or L;

Z₇ represents any amino acid; preferably Z₇=N, S, Y or I;

Z₈ represents any amino acid; preferably Z₈=T, S, Y or A;

X₄ represents 9 amino acids; preferably X₄=Z₁-Z₂-Z₃-Z₄-Z₅-Z₆-Z₇-Z₈-Z₉, wherein

Z₁ represents any amino acid; preferably Z₁=R or I;

Z₂ represents any amino acid; preferably Z₂=K, I, Y, F or L;

Z₃ represents any amino acid; preferably Z₃=G, N, R or Q;

Z₄ represents any amino acid; preferably Z₄=G, H or N;

Z₅ represents any amino acid; preferably Z₅=R or L;

Z₆ represents any amino acid; preferably Z₆=I, L, M, V or R;

Z₇ represents any amino acid; preferably Z₇=Y, W, H or F;

Z₈ represents any amino acid; preferably Z₈=T, R or A;

Z₉ represents any amino acid; preferably Z₉=L, F or R; more preferably Z₉=L or F.

Beta-defensins may be defined as antimicrobial peptides comprising the amino acid sequence:
C-X₁-C-X₂-C-X₃-C-X₄-C-C
wherein

X₁ represents 6 amino acids; preferably X₁=Z₁-Z₂₋Z₃-Z₄-Z₅-Z₆, wherein

Z₁ represents any amino acid; preferably Z₁=R, V or L;

Z₂ represents any amino acid; preferably Z₂=R, I, K or Q;

Z₃ represents any amino acid; preferably Z₃=N or S;

Z₄ represents any amino acid; preferably Z₄=G, K or R;

Z₅ represents any amino acid; preferably Z₅=G;

Z₆ represents any amino acid; preferably Z₆=Q, I, V or F;

X₂ represents 3 or 4 amino acids; preferably X₂ represents 4 amino acids; more preferably X₂=Z₁-Z₂-Z₃-Z4, wherein

Z₁ represents any amino acid; preferably Z₁=L, V, I, H or A;

Z₂ represents any amino acid; preferably Z₂=P or Y;

Z₃ represents any amino acid; preferably Z₃=S, I, N or G;

Z₄ represents any amino acid; preferably Z₄=R, A or S;

X₃ represents 9 amino acids; preferably X₃=Z₁-Z₂-Z₃-Z₄-Z₅-Z₆-Z₇-Z₈-Z₉, wherein

Z₁ represents any amino acid; preferably Z₁=P;

Z₂ represents any amino acid; preferably Z₂=G, I, R or P;

Z₃ represents any amino acid; preferably Z₃=Y, N, P, R, F or H;

Z₄ represents any amino acid; preferably Z₄=T, M or Y;

Z₅ represents any amino acid; preferably Z₅=R or K;

Z₆ represents any amino acid; preferably Z=Q or I;

Z₇ represents any amino acid; preferably Z₇=I or Q;

Z₈ represents any amino acid; preferably Z₈=S;

Z₉ represents any amino acid; preferably Z₉=T;

X₄ represents 6 amino acids; preferably X₄=Z₁-Z₂-Z₃-Z₄-Z₅-Z₆, wherein

Z₁ represents any amino acid; preferably Z₁=Y, F, G or L;

Z₂ represents any amino acid; preferably Z₂=G, H, P, L, R or T;

Z₃ represents any amino acid; preferably Z₃=G, P or R;

Z₄ represents any amino acid; preferably Z=K, P, R, G or Q;

Z₅ represents any amino acid; preferably Z₅=V, A, I or G;

Z₆ represents any amino acid; preferably Z₆=K.

Insect-defensins may be defined as antimicrobial peptides comprising the amino acid sequence:
C-X₁-C-X₂-C-X₃-C-X₄-C-X₅-C
wherein

X₁ represents 5-16 amino acids;

X₂ represents 3 amino acids; preferably X₂=Z₁-Z₂-Z₃, wherein

Z₁ represents any amino acid; preferably Z₁=A or H;

Z₂ represents any amino acid; preferably Z₂=A or R;

Z₃ represents any amino acid; preferably Z₃=H;

X₃ represents 9-11 amino acids;

X4 represents; 4-10 amino acids;

X₅ represents 1 amino acid; preferably X₅=V, T, I, H, K, N or L.

In an embodiment, the defensin of the invention has more than one antimicrobial activity selected from antifungal activity, antibacterial activity and antiviral activity.

A defensin of the invention may be obtained from microorganisms of any genus. For purposes of the present invention, the term “obtained from” as used herein in connection with a given source shall mean that the defensin encoded by a nucleotide sequence is produced by the source or by a strain in which the nucleotide sequence from the source has been inserted. In a preferred aspect, the defensin obtained from a given source is secreted extracellularly.

A defensin of the present invention may be a fungal defensin, and more preferably a yeast defensin such as a Candida, Kluyveromyces, Pichia, Saccharomyces, Schizosaccharomyces, or Yarrowia defensin; or more preferably a filamentous fungal defensin such as an Acremonium, Aspergillus, Aureobasidium, Cryptococcus, Filibasidium, Fusarium, Humicola, Magnaporthe, Mucor, Myceliophthora, Neocallimastix, Neurospora, Paecilomyces, Penicillium, Piromyces, Schizophyllum, Talaromyces, Thermoascus, Thielavia, Tolypocladium, or Trichoderma defensin.

In a preferred aspect, the defensin is a Saccharomyces carlsbergensis, Saccharomyces cerevisiae, Saccharomyces diastaticus, Saccharomyces douglasii, Saccharomyces kluyveri, Saccharomyces norbensis, or Saccharomyces oviformis defensin having antimicrobial activity.

In another preferred aspect, the defensin is an Aspergillus aculeatus, Aspergillus awamori, Aspergillus fumigatus, Aspergillus foetidus, Aspergillus japonicus, Aspergillus nidulans, Aspergillus niger, Aspergillus oryzae, Fusarium bactridioides, Fusarium cerealis, Fusarium crookwellense, Fusarium culmorum, Fusarium graminearum, Fusarium graminum, Fusarium heterosporum, Fusarium negundi, Fusarium oxysporum, Fusarium reticulatum, Fusarium roseum, Fusarium sambucinum, Fusarium sarcochroum, Fusarium sporotrichioides, Fusarium sulphureum, Fusarium torulosum, Fusarium trichothecioides, Fusarium venenatum, Humicola insolens, Humicola lanuginosa, Mucor miehei, Myceliophthora thermophila, Neurospora crassa, Penicillium purpurogenum, Trichoderma harzianum, Trichoderma koningii, Trichoderma longibrachiatum, Trichoderma reesei, or Trichoderma viride defensin.

It will be understood that for the aforementioned species, the invention encompasses both the perfect and imperfect states, and other taxonomic equivalents, e.g., anamorphs, regardless of the species name by which they are known. Those skilled in the art will readily recognize the identity of appropriate equivalents.

Strains of these species are readily accessible to the public in a number of culture collections, such as the American Type Culture Collection (ATCC), Deutsche Sammlung von Mikroorganismen und Zelikulturen GmbH (DSM), Centraalbureau Voor Schimmelcultures (CBS), and Agricultural Research Service Patent Culture Collection, Northern Regional Research Center (NRRL).

Furthermore, such defensins may be identified and obtained from other sources including microorganisms isolated from nature (e.g., soil, composts, water, etc.) using the above-mentioned probes. Techniques for isolating microorganisms from natural habitats are well known in the art. The polynucleotide may then be obtained by similarly screening a genomic or cDNA library of another microorganism. Once a polynucleotide sequence encoding a defensin has been detected with the probe(s), the polynucleotide can be isolated or cloned by utilizing techniques which are well known to those of ordinary skill in the art (see, e.g., Sambrook et al., Molecular Cloning: A Laboratory Manual. 2nd edition. Cold Spring Harbor Laboratory Press, 1989. ISBN 0879693096).

Defensins of the present invention also include fused defensins or cleavable fusion defensins in which another defensin is fused at the N-terminus or the C-terminus of the defensin or fragment thereof. A fused defensin is produced by fusing a nucleotide sequence (or a portion thereof) encoding another defensin to a nucleotide sequence (or a portion thereof) of the present invention. Techniques for producing fusion defensins are known in the art, and include ligating the coding sequences encoding the defensins so that they are in frame and that expression of the fused defensin is under control of the same promoter(s) and terminator.

Nucleic Acid Sequences

The present invention also relates to polynucleotides having a nucleotide sequence which encodes a defensin of the invention.

Examples of such polynucleotides include, but are not limited to, those disclosed in PCT application WO 99/53053, which are hereby incorporated by reference.

In a preferred embodiment, the nucleotide sequence is set forth in SEQ ID NO: 1, SEQ ID NO: 3, SEQ ID NO: 5, SEQ ID NO: 7, SEQ ID NO: 9, SEQ ID NO: 11, SEQ ID NO: 13 or SEQ ID NO: 15 of the present invention. In another preferred embodiment, the nucleotide sequence is the mature defensin coding region of SEQ ID NO: 1, SEQ ID NO: 3, SEQ ID NO: 5, SEQ ID NO: 7, SEQ ID NO: 9, SEQ ID NO: 11, SEQ ID NO: 13 or SEQ ID NO: 15. The present invention also encompasses nucleotide sequences which encode a defensin having the amino acid sequence of SEQ ID NO: 2, SEQ ID NO: 4, SEQ ID NO: 6, SEQ ID NO: 8, SEQ ID NO: 10, SEQ ID NO: 12, SEQ ID NO: 14 or SEQ ID NO: 16, or the mature defensin thereof, which differ from SEQ ID NO: 1, SEQ ID NO: 3, SEQ ID NO: 5, SEQ ID NO: 7, SEQ ID NO: 9, SEQ ID NO: 11, SEQ ID NO: 13 or SEQ ID NO: 15 by virtue of the degeneracy of the genetic code.

The techniques used to isolate or clone a polynucleotide encoding a defensin are known in the art and include isolation from genomic DNA, preparation from cDNA, or a combination thereof. Cloning of the polynucleotides encoding the defensins of the invention from such genomic DNA can be effected, e.g., by using the well known polymerase chain reaction (PCR) or antibody screening of expression libraries to detect cloned DNA fragments with shared structural features. See, e.g., Innis et al., 1990, PCR: A Guide to Methods and Application, Academic Press, New York. Other nucleic acid amplification procedures such as ligase chain reaction (LCR), ligated activated transcription (LAT) and nucleotide sequence-based amplification (NASBA) may be used. The polynucleotides may be cloned from a strain of Eurotium, Aspergillus, Pseudoplectania, Crassostrea, Mesobuthus or another or related organism and thus, for example, may be an allelic or species variant of the defensin encoding region of the nucleotide sequences.

The present invention also relates to polynucleotides having nucleotide sequences which have a degree of identity to the mature defensin coding sequence of SEQ ID NO: 1, SEQ ID NO: 3, SEQ ID NO: 5, SEQ ID NO: 7, SEQ ID NO: 9, SEQ ID NO: 11, SEQ ID NO: 13 or SEQ ID NO: 15 of at least 60%, preferably at least 65%, more preferably at least 70%, more preferably at least 75%, more preferably at least 80%, more preferably at least 85%, more preferably at least 90%, even more preferably at least 95%, and most preferably at least 97% identity, which encode a defensin antimicrobial polypeptide.

Modification of a nucleotide sequence encoding a defensin of the invention may be necessary for the synthesis of defensins substantially similar to the defensin. The term “substantially similar” to the defensin refers to non-naturally occurring forms of the defensin. These defensins may differ in some engineered way from the defensin isolated from its native source, e.g., artificial variants that differ in specific activity, thermostability, pH optimum, or the like. The variant sequence may be constructed on the basis of the nucleotide sequence presented as the defensin encoding region of SEQ ID NO: 1, SEQ ID NO: 3, SEQ ID NO: 5, SEQ ID NO: 7, SEQ ID NO: 9, SEQ ID NO: 11, SEQ ID NO: 13 or SEQ ID NO: 15 e.g., a subsequence thereof, and/or by introduction of nucleotide substitutions which do not give rise to another amino acid sequence of the defensin encoded by the nucleotide sequence, but which correspond to the codon usage of the host organism intended for production of the enzyme, or by introduction of nucleotide substitutions which may give rise to a different amino acid sequence. For a general description of nucleotide substitution, see, e.g., Ford et al., 1991, Protein Expression and Purification 2: 95-107.

It will be apparent to those skilled in the art that such substitutions can be made outside the regions critical to the function of the molecule and still result in an active defensin. Amino acid residues essential to the activity of the defensin, and therefore preferably not subject to substitution, may be identified according to procedures known in the art, such as site-directed mutagenesis or alanine-scanning mutagenesis (see, e.g., Cunningham and Wells, 1989, Science 244: 1081-1085). In the latter technique, mutations are introduced at every positively charged residue in the molecule, and the resultant mutant molecules are tested for antimicrobial activity to identify amino acid residues that are critical to the activity of the molecule. Sites of interaction can also be determined by analysis of the three-dimensional structure as determined by such techniques as nuclear magnetic resonance analysis, crystallography or photoaffinity labelling (see, e.g., de Vos et al., 1992, Science 255: 306-312; Smith et al., 1992, Journal of Molecular Biology 224: 899-904; Wlodaver et a., 1992, FEBS Letters 309: 59-64).

The present invention also relates to polynucleotides encoding a defensin of the invention, which hybridize under low stringency conditions, preferably medium stringency conditions, more preferably medium-high stringency conditions, even more preferably high stringency conditions, and most preferably very high stringency conditions with (i) the mature peptide encoding nucleotide sequence contained in SEQ ID NO: 1, SEQ ID NO: 3, SEQ ID NO: 5, SEQ ID NO: 7, SEQ ID NO: 9, SEQ ID NO: 11, SEQ ID NO: 13 or SEQ ID NO: 15, (ii) the cDNA sequence contained in SEQ ID NO: 1, SEQ ID NO: 3, SEQ ID NO: 5, SEQ ID NO: 7, SEQ ID NO: 9, SEQ ID NO: 11, SEQ ID NO: 13 or SEQ ID NO: 15, or (iii) a complementary strand of (i) or (ii); or allelic variants and subsequences thereof (Sambrook et al., 1989, supra), as defined herein.

The present invention also relates to polynucleotides obtained by (a) hybridizing a population of DNA under low, medium, medium-high, high, or very high stringency conditions with (i) the mature defensin encoding nucleotide sequence contained in SEQ ID NO: 1, SEQ ID NO: 3, SEQ ID NO: 5, SEQ ID NO: 7, SEQ ID NO: 9, SEQ ID NO: 11, SEQ ID NO: 13 or SEQ ID NO: 15, (ii) the cDNA sequence contained in SEQ ID NO: 1, SEQ ID NO: 3, SEQ ID NO: 5, SEQ ID NO: 7, SEQ ID NO: 9, SEQ ID NO: 11, SEQ ID NO: 13 or SEQ ID NO: 15, or (iii) a complementary strand of (i) or (ii); and (b) isolating the hybridizing polynucleotide, which encodes a polypeptide having antimicrobial activity.

Nucleic Acid Constructs

The present invention also relates to nucleic acid constructs comprising a polynucleotide encoding a defensin of the invention operably linked to one or more control sequences which direct the expression of the coding sequence in a suitable host cell under conditions compatible with the control sequences.

A polynucleotide encoding a defensin of the invention may be manipulated in a variety of ways to provide for expression of the defensin. Manipulation of the polynucleotide's sequence prior to its insertion into a vector may be desirable or necessary depending on the expression vector. The techniques for modifying polynucleotide sequences utilizing recombinant DNA methods are well known in the art.

The control sequence may be an appropriate promoter sequence, a nucleotide sequence which is recognized by a host cell for expression of a polynucleotide encoding a defensin of the invention. The promoter sequence contains transcriptional control sequences which mediate the expression of the defensin. The promoter may be any nucleotide sequence which shows transcriptional activity in the host cell of choice including mutant, truncated, and hybrid promoters, and may be obtained from genes encoding extracellular or intracellular polypeptides either homologous or heterologous to the host cell.

Examples of suitable promoters for directing the transcription of the nucleic acid constructs of the present invention in a filamentous fungal host cell are promoters obtained from the genes for Aspergillus oryzae TAKA amylase, Rhizomucor miehei aspartic proteinase, Aspergillus niger neutral alpha-amylase, Aspergillus niger acid stable alpha-amylase, Aspergillus niger or Aspergillus awamori glucoamylase (glaA), Rhizomucor miehei lipase, Aspergillus oryzae alkaline protease, Aspergillus oryzae triose phosphate isomerase, Aspergillus nidulans acetamidase, Fusarium venenatum amyloglucosidase (WO 00/56900), Fusarium venenatum Daria (WO 00/56900), Fusarium venenatum Quinn (WO 00/56900), Fusarium oxysporum trypsin-like protease (WO 96/00787), Trichoderma reesei beta-glucosidase, Trichoderma reesei cellobiohydrolase I, Trichoderma reesei endoglucanase I, Trichoderma reesei endoglucanase II, Trichoderma reesei endoglucanase III, Trichoderma reesei endoglucanase IV, Trichoderma reesei endoglucanase V, Trichoderma reesei xylanase I, Trichoderma reesei xylanase II, Trichoderma reesei beta-xylosidase, as well as the NA2-tpi promoter (a hybrid of the promoters from the genes for Aspergillus niger neutral alpha-amylase and Aspergillus oryzae triose phosphate isomerase); and mutant, truncated, and hybrid promoters thereof.

The control sequence may also be a suitable transcription terminator sequence, a sequence recognized by a host cell to terminate transcription. The terminator sequence is operably linked to the 3′ terminus of the nucleotide sequence encoding the defensin. Any terminator which is functional in the host cell of choice may be used in the present invention.

Preferred terminators for filamentous fungal host cells are obtained from the genes for Aspergillus oryzae TAKA amylase, Aspergillus niger glucoamylase, Aspergillus nidulans anthranilate synthase, Aspergillus niger alpha-glucosidase, and Fusarium oxysporum trypsin-like protease.

The control sequence may also be a suitable leader sequence, a nontranslated region of an mRNA which is important for translation by the host cell. The leader sequence is operably linked to the 5′ terminus of the nucleotide sequence encoding the defensin. Any leader sequence that is functional in the host cell of choice may be used in the present invention.

Preferred leaders for filamentous fungal host cells are obtained from the genes for Aspergillus oryzae TAKA amylase and Aspergillus nidulans triose phosphate isomerase.

The control sequence may also be a polyadenylation sequence, a sequence operably linked to the 3′ terminus of the nucleotide sequence and which, when transcribed, is recognized by the host cell as a signal to add polyadenosine residues to transcribed mRNA. Any polyadenylation sequence which is functional in the host cell of choice may be used in the present invention.

Preferred polyadenylation sequences for filamentous fungal host cells are obtained from the genes for Aspergillus oryzae TAKA amylase, Aspergillus niger glucoamylase, Aspergillus nidulans anthranilate synthase, Fusarium oxysporum trypsin-like protease, and Aspergillus niger alpha-glucosidase.

The control sequence may also be a signal peptide coding region that codes for an amino acid sequence linked to the amino terminus of a defensin and directs the encoded defensin into the cell's secretory pathway. The 5′ end of the coding sequence of the nucleotide sequence may inherently contain a signal peptide coding region naturally linked in translation reading frame with the segment of the coding region which encodes the secreted defensin. Alternatively, the 5′ end of the coding sequence may contain a signal peptide coding region which is foreign to the coding sequence. The foreign signal peptide coding region may be required where the coding sequence does not naturally contain a signal peptide coding region. Alternatively, the foreign signal peptide coding region may simply replace the natural signal peptide coding region in order to enhance secretion of the defensin. However, any signal peptide coding region which directs the expressed defensin into the secretory pathway of a host cell of choice may be used in the present invention.

Effective signal peptide coding regions for filamentous fungal host cells are the signal peptide coding regions obtained from the genes for Aspergillus oryzae TAKA amylase, Aspergillus niger neutral amylase, Aspergillus niger glucoamylase, Rhizomucor miehei aspartic proteinase, Humicola insolens cellulase, and Humicola lanuginosa lipase.

In a preferred aspect, the signal peptide coding region is nucleotides 1 to 69 of SEQ ID NO: 1 which encode amino acids −55 to −33 of SEQ ID NO: 2; nucleotides 1 to 60 of SEQ ID NO: 3 which encode amino acids −48 to −29 of SEQ ID NO: 4; nucleotides 1 to 60 of SEQ ID NO: 5 which encode amino acids −50 to −31 of SEQ ID NO: 6; nucleotides 1 to 66 of SEQ ID NO: 7 which encode amino acids −22 to −1 of SEQ ID NO: 8; nucleotides 1 to 72 of SEQ ID NO: 9 which encode amino acids −24 to −1 of SEQ ID NO: 10; nucleotides 1 to 66 of SEQ ID NO: 11 which encode amino acids −22 to −1 of SEQ ID NO: 12; nucleotides 1 to 66 of SEQ ID NO: 13 which encode amino acids −22 to −1 of SEQ ID NO: 14; or nucleotides 1 to 54 of SEQ ID NO: 15 which encode amino acids −26 to −9 of SEQ ID NO: 16.

The control sequence may also be a propeptide coding region that codes for an amino acid sequence positioned at the amino terminus of a polypeptide. The resultant polypeptide is known as a propolypeptide (or a zymogen in some cases). A propolypeptide is generally inactive and can be converted to a mature active polypeptide by catalytic or autocatalytic cleavage of the propeptide from the propolypeptide. The propeptide coding region may be obtained from the genes for Bacillus subtilis alkaline protease (aprE), Bacillus subtilis neutral protease (nprT), Saccharomyces cerevisiae alpha-factor, Rhizomucor miehei aspartic proteinase, and Myceliophthora thermophila laccase (WO 95/33836).

In a preferred aspect, the propeptide coding region is nucleotides 70 to 165 of SEQ ID NO: 1 which encode amino acids −32 to −1 of SEQ ID NO: 2; nucleotides 61 to 144 of SEQ ID NO: 3 which encode amino acids −28 to −1 of SEQ ID NO: 4; nucleotides 61 to 150 of SEQ ID NO: 5 which encode amino acids −30 to −1 of SEQ ID NO: 6; or nucleotides 55 to 78 of SEQ ID NO: 15 which encode amino acids −8 to −1 of SEQ ID NO: 16.

Where both signal peptide and propeptide regions are present at the amino terminus of a polypeptide, the propeptide region is positioned next to the amino terminus of a polypeptide and the signal peptide region is positioned next to the amino terminus of the propeptide region.

It may also be desirable to add regulatory sequences which allow the regulation of the expression of the defensin relative to the growth of the host cell. Examples of regulatory systems are those which cause the expression of the gene to be turned on or off in response to a chemical or physical stimulus, including the presence of a regulatory compound. Regulatory systems in prokaryotic systems include the lac, tac, and trp operator systems. In yeast, the ADH2 system or GAL1 system may be used. In filamentous fungi, the TAKA alpha-amylase promoter, Aspergillus niger glucoamylase promoter, and Aspergillus oryzae glucoamylase promoter may be used as regulatory sequences. Other examples of regulatory sequences are those which allow for gene amplification. In eukaryotic systems, these include the dihydrofolate reductase gene which is amplified in the presence of methotrexate, and the metallothionein genes which are amplified with heavy metals. In these cases, the nucleotide sequence encoding the defensin would be operably linked with the regulatory sequence.

Expression Vectors

The present invention also relates to recombinant expression vectors comprising a polynucleotide encoding a defensin of the invention, a promoter, and transcriptional and translational stop signals. The various nucleic acids and control sequences described above may be joined together to produce a recombinant expression vector which may include one or more convenient restriction sites to allow for insertion or substitution of the nucleotide sequence encoding the defensin at such sites. Alternatively, a nucleotide sequence encoding a defensin of the invention may be expressed by inserting the nucleotide sequence or a nucleic acid construct comprising the sequence into an appropriate vector for expression. In creating the expression vector, the coding sequence is located in the vector so that the coding sequence is operably linked with the appropriate control sequences for expression.

The recombinant expression vector may be any vector (e.g., a plasmid or virus) which can be conveniently subjected to recombinant DNA procedures and can bring about expression of the nucleotide sequence. The choice of the vector will typically depend on the compatibility of the vector with the host cell into which the vector is to be introduced. The vectors may be linear or closed circular plasmids.

The vector may be an autonomously replicating vector, i.e., a vector which exists as an extrachromosomal entity, the replication of which is independent of chromosomal replication, e.g., a plasmid, an extrachromosomal element, a minichromosome, or an artificial chromosome. The vector may contain any means for assuring self-replication. Alternatively, the vector may be one which, when introduced into the host cell, is integrated into the genome and replicated together with the chromosome(s) into which it has been integrated. Furthermore, a single vector or plasmid or two or more vectors or plasmids which together contain the total DNA to be introduced into the genome of the host cell, or a transposon may be used.

The vectors used for expression of the defensins of the invention preferably contain one or more selectable markers which permit easy selection of transformed cells. A selectable marker is a gene the product of which provides for biocide or viral resistance, resistance to heavy metals, prototrophy to auxotrophs, and the like.

Examples of selectable markers for use in a filamentous fungal host cell include, but are not limited to, amdS (acetamidase), argB (ornithine carbamoyltransferase), bar (phosphinothricin acetyltransferase), hph (hygromycin phosphotransferase), niaD (nitrate reductase), pyrg (orotidine-5′-phosphate decarboxylase), sC (sulfate adenyltransferase), and trpC (anthranilate synthase), as well as equivalents thereof. Preferred for use in an Aspergillus cell are the amdS and pyrg genes of Aspergillus nidulans or Aspergillus oryzae and the bar gene of Streptomyces hygroscopicus.

The vectors preferably contain an element(s) that permits integration of the vector into the host cell's genome or autonomous replication of the vector in the cell independent of the genome.

For integration into the host cell genome, the vector may rely on the polynucleotide's sequence encoding the defensin or any other element of the vector for integration into the genome by homologous or nonhomologous recombination. Alternatively, the vector may contain additional nucleotide sequences for directing integration by homologous recombination into the genome of the host cell at a precise location(s) in the chromosome(s). To increase the likelihood of integration at a precise location, the integrational elements should preferably contain a sufficient number of nucleic acids, such as 100 to 10,000 base pairs, preferably 400 to 10,000 base pairs, and most preferably 800 to 10,000 base pairs, which have a high degree of identity with the corresponding target sequence to enhance the probability of homologous recombination. The integrational elements may be any sequence that is homologous with the target sequence in the genome of the host cell. Furthermore, the integrational elements may be non-encoding or encoding nucleotide sequences. On the other hand, the vector may be integrated into the genome of the host cell by non-homologous recombination.

For autonomous replication, the vector may further comprise an origin of replication enabling the vector to replicate autonomously in the host cell in question. The origin of replication may be any plasmid replicator mediating autonomous replication which functions in a cell. The term “origin of replication” or “plasmid replicator” is defined herein as a nucleotide sequence that enables a plasmid or vector to replicate in vivo.

Examples of origins of replication useful in a filamentous fungal cell are AMA1 and ANS1 (Gems et al., 1991, Gene 98: 61-67; Cullen et al., 1987, Nucleic Acids Research 15: 9163-9175; WO 00/24883). Isolation of the AMA1 gene and construction of plasmids or vectors comprising the gene can be accomplished according to the methods disclosed in WO 00/24883.

More than one copy of a polynucleotide encoding a defensin of the invention may be inserted into the host cell to increase production of the gene product. An increase in the copy number of the polynucleotide can be obtained by integrating at least one additional copy of the sequence into the host cell genome or by including an amplifiable selectable marker gene with the polynucleotide where cells containing amplified copies of the selectable marker gene, and thereby additional copies of the polynucleotide, can be selected for by cultivating the cells in the presence of the appropriate selectable agent.

The procedures used to ligate the elements described above to construct the recombinant expression vectors of the present invention are well known to one skilled in the art (see, e.g., Sambrook et al., 1989, supra).

Filamentous Fungal Host Cells

The host cell (or organism) of the invention is a filamentous fungus including all filamentous forms of the divisions Ascomycota, Basidiomycota, Chytridiomycota and Zygomycota (as defined by Kirk, P. M. et al., In, Ainsworth and Bisby's Dictionary of The Fungi, 9th edition, 2001, CAB International, Wallingford, UK). The filamentous fungi are characterized by a vegetative mycelium composed of chitin and glucan and/or other complex polysaccharides. Vegetative growth is by hyphal elongation. Preferably carbon catabolism is obligately aerobic.

In an embodiment, the filamentous fungal host cell (or organism) belongs to the order Eurotiales of the subdivision Ascomycota; preferably it more specifically belongs to the Trichocomaceae family.

In a more preferred embodiment, the filamentous fungal host cell (or organism) is a cell of a species of, but not limited to, Acremonium, Aspergillus, Emericella, Eurotium, Fusarium, Humicola, Mucor, Myceliophthora, Neurospora, Penicillium, Eupenicillium, Thielavia, Tolypocladium, and Trichoderma or a teleomorph, anamorph or synonym thereof. In an even more preferred embodiment, the filamentous fungal host cell is an Aspergillus. In another even more preferred embodiment, the filamentous fungal host cell is an Acremonium. In another even more preferred embodiment, the filamentous fungal host cell is a Fusarium. In another even more preferred embodiment, the filamentous fungal host cell is a Humicola. In another even more preferred embodiment, the filamentous fungal host cell is a Mucor. In another even more preferred embodiment, the filamentous fungal host cell is a Myceliophthora. In another even more preferred embodiment, the filamentous fungal host cell is a Neurospora. In another even more preferred embodiment, the filamentous fungal host cell is a Penicillium. In another even more preferred embodiment, the filamentous fungal host cell is a Thielavia. In another even more preferred embodiment, the filamentous fungal host cell is a Tolypocladium. In another even more preferred embodiment, the filamentous fungal host cell is a Trichoderma. In a most preferred embodiment, the filamentous fungal host cell is an Aspergillus awamori, Aspergillus foetidus, Aspergillus japonicus, Aspergillus aculeatus, Aspergillus niger or Aspergillus oryzae. In another preferred embodiment, the filamentous fungal host cell is a Fusarium of the section Discolor (also known as the section Fusarium). For example, the filamentous fungal host cell may be a Fusarium bactridioides, Fusarium cerealis, Fusarium crookwellense, Fusarium culmorum, Fusarium graminearum, Fusarium graminum, Fusarium heterosporum, Fusarium negundi, Fusarium reticulatum, Fusarium roseum, Fusarium sambucinum, Fusarium sarcochroum, Fusarium sulphureum, or Fusarium trichothecioides. In another prefered embodiment, the filamentous fungal host cell is a Fusarium strain of the section Elegans, e.g., Fusarium oxysporum. In another most preferred embodiment, the filamentous fungal host cell is a Humicola insolens or Humicola lanuginosa. In another most preferred embodiment, the filamentous fungal host cell is a Mucor miehei. In another most preferred embodiment, the filamentous fungal host cell is a Myceliophthora thermophilum. In another most preferred embodiment, the filamentous fungal host cell is a Neurospora crassa cell. In another most preferred embodiment, the filamentous fungal host cell is a Penicillium purpurogenum or Penicillium funiculosum (WO 00/68401). In another most preferred embodiment, the filamentous fungal host cell is a Thielavia terrestris. In another most preferred embodiment, the Trichoderma cell is a Trichoderma harzianum, Trichoderma koningii, Trichoderma longibrachiatum, Trichoderma reesei or Trichoderma viride.

In a particular embodiment the filamentous host cell is an Aspergillus oryzae or Aspergillus niger.

In a preferred embodiment of the invention the host cell is a protease deficient or protease minus strain.

This may, e.g., be the protease deficient strain Aspergillus oryzae JaL 125 having the alkaline protease gene named “alp” deleted. This strain is described in WO 9735956 (Novo Nordisk A/S) 02.10.1997, or EP 429490 (Genencor) 05.06.1991, or the TPAP free host cell, in particular a strain of Aspergillus niger, disclosed in WO 96/14404 (Novo Nordisk A/S). Further, also host cells, especially A. niger or A. oryzae, with reduced production of the transcriptional activator (prtT) as described in WO 01/68864 (Novozymes A/S) is specifically contemplated according to the invention.

Introns

Eukaryotic genes may be interrupted by intervening sequences (introns) which must be modified in precursor transcripts in order to produce functional mRNAs. This process of intron removal is known as pre-mRNA splicing. Usually, a branchpoint sequence of an intron is necessary for intron splicing through the formation of a lariat. Signals for splicing reside directly at the boundaries of the intron splice sites. The boundaries of intron splice sites usually have the consensus intron sequences GT and AG at their 5′ and 3′ extremities, respectively. While no 3′ splice sites other than AG have been reported, there are reports of a few exceptions to the 5′ GT splice site. For example, there are precedents where CT or GC is substituted for GT at the 5′ boundary. There is also a strong preference for the nucleotide bases ANGT to follow GT where N is A, C, G, or T (primarily A or T in Saccharomyces species), but there is no marked preference for any particular nucleotides to precede the GT splice site. The 3′ splice site AG is primarily preceded by a pyrimidine nucleotide base (Py), i.e., C or T.

The number of introns that can interrupt a fungal gene ranges from one to two, three, four, five, six, seven, eight, nine, ten, eleven, twelve or more introns. They may be distributed throughout a gene or situated towards the 5′ or 3′ end of a gene. In Saccharomyces cerevisiae, introns are located primarily at the 5′ end of the gene. Introns may be generally less than 1 kb in size, and usually are less than 400 bp in size in yeast and less than 100 bp in filamentous fungi.

The Saccharomyces cerevisiae intron branchpoint sequence 5′-TACTAAC-3′ rarely appears exactly in filamentous fungal introns. Sequence stretches closely or loosely resembling TACTMC are seen at equivalent points in filamentous fungal introns with a general consensus NRCTRAC where N is A, C, G, or T, and R is A or G. For example, the fourth position T is invariant in both the Neurospora crassa and Aspergillus nidulans putative consensus sequences. Furthermore, nucleotides G, A, and C predominate in over 80% of the positions 3, 6, and 7, respectively, although position 7 in Aspergillus nidulans is more flexible with only 65% C. However, positions 1, 2, 5, and 8 are much less strict in both Neurospora crassa and Aspergillus nidulans. Other filamentous fungi have similar branchpoint stretches at equivalent positions in their introns, but the sampling is too small to discern any definite trends.

Methods and Uses

In a first aspect, the present invention provides a recombinant filamentous fungal host cell, comprising a nucleic acid construct, which nucleic acid construct comprises a foreign nucleic acid sequence encoding a defensin and one or more intron sequence(s). The term “foreign nucleic acid sequence” is intended to mean a nucleic acid sequence which has been introduced from the outside (from a foreign source).

The filamentous fungal host cell may be capable of expressing (producing) the defensin in an amount of at least 150%, preferably 200%, more preferably 250%, and most preferably 300% of the amount obtained when using a nucleic acid construct without an intron sequence, such as a cDNA sequence.

The filamentous fungal host cell may be grown in YPM growth medium for 3-5 days at 30-35 degrees Celsius under suitable agitation. This and other suitable methods for cultivating filamentous fungi are well-known in the art. The filamentous fungal host cell may be used for recombinant production of defensins.

In a second aspect the invention provides a method for recombinant production of a defensin in a filamentous fungal host cell, which includes cultivating the filamentous fungal host cell comprising a nucleic acid construct, which nucleic acid construct comprises a nucleic acid sequence encoding the defensin peptide and one or more intron sequence(s); and recovering the defensin peptide. Suitable recovery methods are well-known in the art.

The invention also relates to use of a nucleic acid construct, which comprises a nucleic acid sequence encoding a defensin peptide and one or more intron sequence(s), for improving the recombinant expression level of the defensin in a filamentous fungal host cell.

The expression level of the defensin may be at least 50% higher, preferably at least 75% higher, more preferably at least 100% higher, even more preferably at least 125% higher, most preferably 150% higher, and in particular 200% higher compared to using a nucleic acid construct which does not comprise an intron sequence, such as a cDNA sequence. Alternatively, the expression level of the defensin may be at least 150%, preferably 200%, more preferably 250%, and most preferably 300% of the expression level obtained when using a nucleic acid construct without an intron sequence, such as a cDNA sequence. The expression level is measured in gram defensin protein per liter fermentation fluid.

In an embodiment, the nucleic acid construct of the invention contains 1, 2, 3, 4, or 5 intron sequence(s), preferably 1, 2, 3 or 4 intron sequence(s), more preferably 1, 2 or 3 intron sequence(s), even more preferably 1 or 2 intron sequence(s), and most preferably one intron sequence.

In another embodiment, the nucleic acid construct of the invention comprises a nucleic acid sequence encoding a defensin peptide and at least one, preferably at least two, more preferably at least three, and most preferably at least four intron sequences.

The intron sequence(s) may be located in the signal-, pro- or mature peptide encoding part of the nucleic acid construct of the invention. When the nucleic acid construct contains more than one intron, the introns can be located in different parts of the construct.

The intron sequence(s) may in fact be located in any part of the defensin gene which is transcribed into mRNA.

Pharmaceutical Formulations

The defensins of this invention can be incorporated into a variety of pharmaceutical formulations for therapeutic administration. More particularly, the defensins of the present invention can be formulated into pharmaceutical compositions by combination with appropriate, pharmaceutically acceptable carriers or diluents, and may be formulated into preparations in solid, semi-solid, liquid or gaseous forms, such as tablets, capsules, powders, granules, ointments, creams, foams, solutions, suppositories, injections, inhalants, gels, microspheres, lotions, and aerosols. As such, administration of the defensins can be achieved in various ways, including oral, buccal, rectal, parenteral, intraperitoneal, intradermal, transdermal, intracheal, etc., administration. According to invention, the defensins are systemic after administration.

The defensins of the invention can be administered alone, in combination with each other, or they can be used in combination with other known compounds (e.g., perforin, anti-inflammatory agents, antibiotics, etc.) In pharmaceutical dosage forms, the defensins may be administered in the form of their pharmaceutically acceptable salts. The following methods and excipients are merely exemplary and are in no way limiting.

For oral preparations, the defensins can be used alone or in combination with appropriate additives to make tablets, powders, granules or capsules, for example, with conventional additives, such as lactose, mannitol, corn starch or potato starch; with binders, such as crystalline cellulose, cellulose derivatives, acacia, corn starch or gelatins; with disintegrators, such as corn starch, potato starch or sodium carboxymethylcellulose; with lubricants, such as talc or magnesium stearate; and if desired, with diluents, buffering agents, moistening agents, preservatives and flavoring agents.

The defensins can be formulated into preparations for injections by dissolving, suspending or emulsifying them in an aqueous or nonaqueous solvent, such as vegetable or other similar oils, synthetic aliphatic acid glycerides, esters of higher aliphatic acids or propylene glycol; and if desired, with conventional additives such as solubilizers, isotonic agents, suspending agents, emulsifying agents, stabilizers and preservatives.

The defensins of the invention can be utilized in aerosol formulation to be administered via inhalation. The defensins can be formulated into pressurized acceptable propellants such as dichlorodifluoromethane, propane, nitrogen and the like.

Furthermore, the defensins can be made into suppositories by mixing with a variety of bases such as emulsifying bases or water-soluble bases. The defensins can be administered rectally via a suppository. The suppository can include vehicles such as cocoa butter, carbowaxes and polyethylene glycols, which melt at body temperature, yet are solidified at room temperature.

Unit dosage forms for oral or rectal administration such as syrups, elixirs, and suspensions may be provided wherein each dosage unit, for example, teaspoonful, tablespoonful, tablet or suppository, contains a predetermined amount of the composition containing one or more defensins of the present invention. Similarly, unit dosage forms for injection or intravenous administration may comprise the defensins of the present invention in a composition as a solution in sterile water, normal saline or another pharmaceutically acceptable carrier.

The term “unit dosage form”, as used herein, refers to physically discrete units suitable as unitary dosages for human and animal subjects, each unit containing a predetermined quantity of defensins of the invention calculated in an amount sufficient to produce the desired effect in association with a pharmaceutically acceptable diluent, carrier or vehicle. The specifications for the unit dosage forms of the present invention depend on the particular defensin employed and the effect to be achieved, and the pharmacodynamics associated with the defensin in the host.

The pharmaceutically acceptable excipients, such as vehicles, adjuvants, carriers or diluents, are readily available to the public. Moreover, pharmaceutically acceptable auxiliary substances, such as pH adjusting and buffering agents, tonicity adjusting agents, stabilizers, wetting agents and the like, are readily available to the public.

Typical dosages for systemic administration range from 0.1 pg to 100 milligrams per kg weight of subject per administration. A typical dosage may be one tablet taken from two to six times daily, or one time-release capsule or tablet taken once a day and containing a proportionally higher content of active ingredient. The time-release effect may be obtained by capsule materials that dissolve at different pH values, by capsules that release slowly by osmotic pressure, or by any other known means of controlled release.

Those of skill will readily appreciate that dose levels, can vary as a function of the specific defensin, the severity of the symptoms and the susceptibility of the subject to side effects. Some of the specific defensins are more potent than others. Preferred dosages for a given defensin are readily determinable by those of skill in the art by a variety of means. A preferred means is to measure the physiological potency of a given defensin.

The use of liposomes as a delivery vehicle is one method of interest. The liposomes fuse with the cells of the target site and deliver the contents of the lumen intracellularly. The liposomes are maintained in contact with the cells for sufficient time for fusion, using various means to maintain contact, such as isolation, binding agents, and the like. In one aspect of the invention, liposomes are designed to be aerosolized for pulmonary administration. Liposomes may be prepared with purified proteins or peptides that mediate fusion of membranes, such as Sendai virus or influenza virus, etc. The lipids may be any useful combination of known liposome forming lipids, including cationic or zwitterionic lipids, such as phosphatidylcholine. The remaining lipid will normally be neutral or acidic lipids, such as cholesterol, phosphatidyl serine, phosphatidyl glycerol, and the like.

For preparing the liposomes, the procedure described by KATO, et al. Expression of hepatitis B virus surface antigen in adult rat liver. Co-introduction of DNA and nuclear protein by a simplified liposome method (J. Biol. Chem., February 1991, 266: 3361-3364) may be used. Briefly, the lipids and lumen composition containing peptides are combined in an appropriate aqueous medium, conveniently a saline medium where the total solids will be in the range of about 1-10 weight percent. After intense agitation for short periods of time, from about 5-60 sec., the tube is placed in a warm water bath, from about 2540° C. and this cycle repeated from about 5-10 times. The composition is then sonicated for a convenient period of time, generally from about 1-10 sec. and may be further agitated by vortexing. The volume is then expanded by adding aqueous medium, generally increasing the volume by about from 1-2 fold, followed by shaking and cooling. This method allows for the incorporation into the lumen of high molecular weight molecules.

Formulations with Other Active Agents

For use in the subject methods, the defensins of the invention may be formulated with other pharmaceutically active agents (such as steroids), which are well-known in the art, particularly other antimicrobial agents. Other agents of interest include a wide variety of antibiotics, as known in the art. Classes of antibiotics include penicillins, e.g., penicillin G, penicillin V, methicillin, oxacillin, carbenicillin, nafcillin, ampicillin, etc.; penicillins in combination with beta-lactamase inhibitors, cephalosporins, e.g., cefaclor, cefazolin, cefuroxime, moxalactam, etc.; carbapenems; monobactams; aminoglycosides; tetracyclines; macrolides; lincomycins; polymyxins; sulfonamides; quinolones; cloramphenical; metronidazole; spectinomycin; trimethoprim; vancomycin; etc.

Anti-mycotic agents are also useful, including polyenes, e.g., amphotericin B, nystatin; 5-flucosyn; and azoles, e.g., miconazol, ketoconazol, itraconazol and fluconazol. Antituberculotic drugs include isoniazid, ethambutol, streptomycin and rifampin. Cytokines may also be included in a formulation of the defensins of the invention, e.g., interferon gamma, tumor necrosis factor alpha, interleukin 12, etc.

The present invention is further described by the following examples which should not be construed as limiting the scope of the invention.

### EXAMPLES

Chemicals used as buffers and substrates were commercial products of at least reagent grade.

### Example 1

### Expression of Intron Containing Defensin Encoding Sequence in Aspergillus oryzae

The 361 bp BamH1-Xho1 digested PCR product amplified from a P. nigrella cDNA library (SEQ ID NO: 6 from WO 03/044049 (Novozymes A/S)) was cloned into an Aspergillus expression vector, as previously described in WO 03/044049, to give plasmid pMT2549. The sequence of the intron in the Plectasin encoding sequence of pMT2549 was modified by using standard in vitro mutagenesis and SOE to introduce a single extra base thereby creating a restriction site within the intron. The resulting intron-containing (59 bp) Plectasin encoding sequence is shown as SEQ ID NO: 17. The corresponding expression plasmid was named pMT2647.

pMT2647 was transformed into Aspergillus oryzae BECh2 as previously described in WO 03/044049. In the first place, 14 individual transformants were twice reisolated, grown on YPM medium (1% yeast extract, 2% bacto peptone and 2% maltose) and finally 10 micro-L samples were analysed on SDS gels as previously described in WO 03/044049. For some of the transformants the amount of plectasin produced appeared from the staining intensities to considerably surpass the estimated 50 mg/L maximal level previously obtained under these growth conditions for transformants of A. oryzae with the expression plasmid encoding the intron-free cDNA encoding Plectasin (see WO 03/044049).

While several of the 14 pMT2549 transformants appeared to produce higher levels of Plectasin than did the best of the 30 intron-less transformants previously analysed in WO 03/044049, it should be kept in mind that each acetamide selected transformant represents an individual transformation and integration event resulting in considerable differences in yields between individual transformants. Such yield differences are well known to occur in systems based on non-homologous recombination, and are generally considered to be a consequence of the random integration locus and the number of expression plasmid integrated. It was therefore decided to compare expression also from a single copy of an expression vector integrated at a defined locus (see Example 2).

### Example 2

### Expression of Defensin in Aspergillus oryzae from Defined Single Copy Integrants

To compare directly the expression in A. oryzae of Plectasin from intron-less cDNA to expression from the intron-containing Plectasin encoding sequence, these were transferred from pMT2548 (see WO 03/044049) and pMT2549 (above), respectively, on approx. 1.1 kb BamH1-Xba1 fragments to the BamH1-Xba1 fragment of a vector based on pJaL485 (8.3 kb). (see Example 3 in WO 03/008575 (Novozymes A/S)). pJaL485 contains for selection only the part of the A. oryzae niaD gene encoding the C-terminal part of nitrate reductase. Using a host strain such as JaL507, a derivative of JaL294 (see Example 8 in WO 03/008575) containing a deletion in this part of the niaD gene, a functional nitrate reductase, and thus the ability to grow on nitate as nitrogen source, can be restored only by homologous recombination. It has been found that most transformants selected in this way are indeed single copy integrants resulting from a single homologous cross-over. Tandem integrants at the niaD site do occur at a lower frequency. The intron-less and intron-containing pJaL485 derived expression plasmids were named pMT2777 and pMT2836, respectively. For each of these plasmids 4 transformants of A. oryzae JaL507 were selected and shown by Southern analysis to contain a single copy of the transforming plasmid integrated homologously at the niaD locus. The pMT2777 derived A. oryzae transformants were named MT2882-2885 and the pMT2836 derived transformants were MT2886-2889. Each of the transformants MT2882-2889 were grown on YPM as described above and 10 μL samples from culture supernatants after 3 and after 7 days of growth were analysed by PAGE SDS gels as above. The result clearly shows very little spread among the transformants with each plasmid (as expected since they should be independent but identical strains). On the other hand it is evident that the expression level in MT2886-2889 (intron-containing) is higher than in MT2882-2885 (intron-less).

Relative Plectasin levels in the day 7 samples of MT2882-2889 have also been determined by an ELISA assay (see Example 4).

### Example 3

### Increased Expression of Defensin from Genes Containing Intron at Different Position and/or Different Intron

To facilitate transfer of sequences encoding Plectasin variants from, e.g., E. coli and S. cerevisiae vectors to Aspergillus expression vectors, it was decided to relocate the Plectasin intron from originally being positioned in the sequence encoding mature Plectasin to a position in the prepro-peptide encoding sequence. To do this, mutations were introduced in the intron-less Plectasin encoding sequence by in vitro mutagenesis resulting in a MluN1 site being located in the signal peptide encoding sequence. It was only possible to introduce the MluN1 site by allowing an amino acid change in the signal peptide (Leu17 changed to Ala). This amino acid change is not predicted to impair signal peptide function according to commonly used signal prediction programs. The sequence of the intron-less MluN1 contain-ing, Plectasin encoding sequence is shown as SEQ ID NO: 18; the corresponding Aspergillus expression plasmid was called pMT2898.

An intron derived from the second intron of A. niger glucoamylase and constructed such that it could be excised from a plasmid pMT2374 as a 55 bp SnaB1-Pvu2 (see Example 1 in WO 03/104457 (Novozymes A/S)) was inserted in the MluN1 site of pMT2898 to give pMT2899 in which the intron was verified to be inserted in the proper orientation. The sequence of the intron containing, Plectasin encoding sequence of pMT2899 is shown as SEQ ID NO: 19.

Also, the intron originally present in the sequence encoding mature Plectasin was made synthetically such that it could likewise be moved as a SnaB1-Pvu2 fragment. This was possible only by altering the last but two bases of the intron from a T to a C. This intron was also transferred to the MluN1 site of pMT2898 to give pMT2900 in which the correct intron orientation was verified by sequencing. The sequence of the intron-containing, Plectasin encoding sequence of pMT2900 is shown as SEQ ID NO: 20.

Plasmids pMT2898, 2899 and 2900 were transformed into A. oryzae BECh 2 selecting for growth on acetamide. Approximately 20 transformants with each plasmid were twice reisolated and grown on YPM as described above. From SDS PAGE gels of the supernatants it was evident that the average Plectasin expression level was considerably higher for transformants with the intron containing constructs pMT2899 and pMT2900 compared to the intron-less construct pMT2898. Again, since expression levels between individual acetamide selected transformants vary widely, it was decided to transfer the expression cassettes of MT2898-2900 to the niaD based expression vector derived from pJaL485 as described above in Example 2. The resulting expression plasmids were pMT2901, pMT2902, and pMT2903 corresponding to pMT2898, pMT2899 and pMT2900, respectively. pMT2901-2903 were transformed into the niaD defective host JaL507, as described above in Example 2. A number of transformants were reisolated for each of the transformations. The transformants were grown on YPM and supernatants run on SDS page gels as above. Also in this case it is obvious that the expression level is higher for the intron-containing constructs pMT2902 and pMT2903 than it is for transformants with the construct pMT2901. It remains to be proven by Southern analysis which transformants are indeed single copy integrants, but from experience the majority of nitrate selected transformants in this set up can be shown to be single copy integrants, even if tandem integration does occur. Two transformants were selected as representative strains for each of pMT2901 (strains MT2946 and MT2947), pMT2902 (strains MT2948 and MT2949), and pMT2903 (strains MT2952 and MT2953).

ELISA quantification of Plectasin was also done for MT2946-2949 (see Example 4).

### Example 4

### Competitive ELISA—Estimation of Plectasin yields in Aspergillus oryzae Fermentations

Using rabbit polyclonal antibody generated against purified Plectasin, an indirect competitive ELISA was applied to estimate yields in Aspergillus oryzae fermentation broths. This type of assay is a standard procedure generally applied to quantify amounts of a given protein/peptide, given that an antiserum has been generated.

The following material and buffers were used:
Plectasin—a defensin described in PCT application WO 03/044049;
F96 MaxiSorp Plate (Nunc, Cat. no.: 439454);
F96 Microwell Plate (Nunc, Cat. no.: 269787);
Skim milk powder (MERCK, Cat. no.: 1.15363.0500);
Tween 20 (MERCK, Cat. no.: 8.22184.0500);
Plectasin specific rabbit polyclonal antibody (Novozymes);
Swine Anti-Rabbit Immunoglobulins/HRP (DAKO, P0448);
TMB Plus, Ready-to-Go (KemEnTek, Cat. no.: 4390);
Sulfuric acid (MERCK, Cat. no.: 1.00731.1000).
PBS, pH 7.2, 1 L:
   8.00 g NaCl,
   0.20 g KCl,
   1.04 g K₂HPO₄,
   0.32 g KH₂PO₄;
Blocking buffer: PBS, 2 % Skim milk;
Washing buffer: PBS, 0.05% Tween 20;
Dilution buffer: PBS, 0.5 % Skim milk, 0.05 % Tween 20.

Briefly, undiluted and serial 2-fold dilutions of culture broths were pre-incubated with 1:1000 polyclonal antibody in dilution buffer. Upon incubation for 2 hours at room temperature, these samples were transferred to Plectasin pre-coated plates (coated with 0.1 microgram/ml Plectasin and residual binding blocked using blocking buffer). Upon 1 hour incubation at room temperature, bound antibody was detected using a secondary antibody (Swine anti rabbit—HRP) and thereafter a chromogen (TMB Plus). Detection of Plectasin bound antibody was measured through absorbance at 450 nm, which resulted in a titration curve of antibody binding.

Throughout the assay, plates were washed using washing buffer, and substance dilutions made as described by the manufacturer (DAKO & kemEnTek).

Interpretations: No detection of antibody bound to the well means that complete binding (inhibition) of antibody to unknown fermentation broth has occurred; whereas maximum absorbance measured equals absence of inhibition by the competitor (unknown fermentation broth). In this way we were able to estimate the amounts (diluted broths) that were needed to inhibit 50% of the binding to the wells. The results are depicted in the table below. Results were calculated relatively to yields measured by fermentation broths with no intron (MT2882, MT2883, MT2884 and MT2885). The results are shown in Table 1.

The results show that by using gene constructs containing an intron, the expression levels increased to about 330% of the expression levels obtained by using gene constructs with no intron.

TABLE 1

| Intron | Culture ID | Relative yield | Average yield |
|---|---|---|---|
| − | MT2882, day 7 | 106 | 100 |
| − | MT2883, day 7 | 108 | |
| − | MT2884, day 7 | 96 | |
| − | MT2885, day 7 | 91 | |
| + | MT2886, day 7 | 335 | 330 |
| + | MT2887, day 7 | 368 | |
| + | MT2888, day 7 | 310 | |
| + | MT2889, day 7 | 320 | |
| − | MT2946 | 77 | 89 |
| − | MT2947 | 100 | |
| + | MT2948 | 268 | 331 |
| + | MT2949 | 394 | |

### Example 5

### Increased Expression of Defensin from Eurotium Amstelodami

A defensin encoding cDNA from Eurotium amstelodami was earlier identified and the defensin was named Eurocin (see Examples of PCT/DK2005/000725 (Novozymes A/S); or SEQ ID NO: 3). The cDNA was expressed in A. oryzae to give the active Eurocin defensin peptide. In order to increase the expression level, an expression construct containing the genomic DNA sequence (including two introns) was made.

Genomic DNA from E. amstelodami was prepared using a standard method for preparation of fungal genomic DNA. Approximately 50 ng of genomic DNA was used as template in a PCR reaction with the following primers.

| | |
|---|---|
| Primer A: | |
| TCTTGGATCCACCATGCACTTCACCAAGGTCTCC | (SEQ ID NO: 21) |
| Primer B: | |
| TCTTCTCGAGTTAGAAAGAACAGGTGCAGGTAC | (SEQ ID NO: 22) |

10 pmoles of each primer was used in a 50 microliter reaction volume. The annealing temperature was 55 degrees Celsius and extension at 72 degrees Celsius for 1 minute. A total of 35 cycles were run using the Expand High Fidelity PCR System (Roche). The PCR product was digested with BamH1 and Xho1 which cut in the overhangs introduced by the PCR primers. The digest was run on a 2% agarose gel and a band of approximately 400 bp was isolated. The isolated band was ligated into BamH1-Xho1 digested plasmid pMT2786 (see Example 2 of PCT/DK2005/000725) and transformed into E coli MT173 selecting for leucine prototrophy. The BamH1-Xho1 insert was sequenced and shown to encode the prepro-Eurocin sequence corresponding to the cDNA previously characterized (see PCT/DK2005/000725 or SEQ ID NO: 3), but also containing two intron sequences of 45 and 53 bases, respectively. The sequence and structure of the BamH1-Xho1 insert is shown in SEQ ID NO: 23. The Aspergillus expression plasmid containing the insert was named pMT2945.

pMT2945 was transformed into A. oryzae BECh2 as described in Example 1, and 20 transformants were reisolated and fermented as in Example 1. Supernatants were analyzed on SDS gels also as described above. As previously, parallel experiments were also made using transformants with the Eurocin cDNA based expression plasmid pMT2935 (see Example 2 of PCT/DK2005/000725).

The transformants based on the cDNA construct pMT2935 and the intron containing construct pMT2945 all yielded distinct bands corresponding to Eurocin in size on the SDS gels.

Transformants based on the intron containing genomic construct pMT2945 were estimated to yield 300-400% of the expression level obtained with the transformants based on the cDNA construct pMT2935.

### Example 6

### Using the HMM Files from the PFAM Database to Identify a Defensin

Sequence analysis using hidden markov model profiles (HMM profiles) may be carried out either online on the Internet or locally on a computer using the well-known HMMER freely available software package. The current version is HMMER 2.3.2 from October 2003.

The HMM profiles may be obtained from the well-known PFAM database. The current version is PFAM 16.0 from November 2004. Both HMMER and PFAM are available for all computer platforms from, e.g., Washington University in St. Louis (USA), School of Medicine (http://pfam.wustl.edu and http://hmmer.wustl.edu).

If a query amino acid sequence, or a fragment thereof, belongs to one of the following five PFAM families, the amino acid sequence is a defensin according to the present invention:
Defensin_beta or “Beta Defensin”, accession number: PF00711;
Defensin_propep or “Defensin propeptide”, accession number: PF00879;
Defensin_{—}1 or “Mammalian defensin”, accession number: PF00323;
Defensin_{—}2 or “Arthropod defensin”, accession number: PF01097;
Gamma-thionin or “Gamma-thionins family”, accession number: PF00304.

An amino acid sequence belongs to a PFAM family, according to the present invention, if it generates an E-value which is greater than 0.1, and a score which is larger or equal to zero, when the PFAM database is used online, or when the hmmpfam program (from the HMMER software package) is used locally.

When the sequence analysis is carried out locally using the “hmmpfam” program, it is necessary to obtain (download) the HMM profiles from the PFAM database. Two profiles exist for each family; “xxx_ls.hmm” for glocal searches, and “xxx_fs.hmm” for local searches (“xxx” is the name of the family). That makes a total of ten profiles for the five families mentioned above.

These ten profiles may be used individually, or joined (appended) into a single profile (using a text editor—the profiles are ASCII files) that could be named, e.g., “defensin.hmm”. A query amino acid sequence can then be evaluated by using the following command line:
hmmpfam-E 0.1 defensin.hmm sequence_file
wherein “sequence_file” is a file with the query amino acid sequence in any of the formats recognized by the HMMER software package.

If the score is larger or equal to zero (0.0), and the E-value is greater than 0.1, the query amino acid sequence is a defensin according to the present invention.

The PFAM database is further described in Bateman et al. The Pfam Protein Families Database. Nucleic acids res., January 2004, vol. 32, p.D138-D141.

1. A recombinant filamentous fungal host cell, comprising a nucleic acid construct, which nucleic acid construct comprises a foreign nucleic acid sequence encoding a defensin and one or more intron sequence(s).

2. The host cell of claim 1, which is an Aspergillus host cell.

3. The Aspergillus host cell of claim 2, which is an Aspergillus niger or Aspergillus oryzae host cell.

4. The host cell of claim 1, wherein the defensin is an alpha defensin, beta defensin, theta defensin, arthropod defensin, insect defensin or plant defensin.

5. The host cell of claim 1, which is capable of producing the defensin in an amount of at least 150% of the amount obtained when using a nucleic acid construct without an intron sequence.

6. A method for recombinant production of a defensin in a filamentous fungal host cell, comprising cultivating the filamentous fungal host cell comprising a nucleic acid construct, which nucleic acid construct comprises a nucleic acid sequence encoding the defensin peptide and one or more intron sequence(s); and recovering the defensin peptide.

7. The method of claim 6, wherein the filamentous fungal host cell is an Aspergillus host cell.

8. The method of claim 7, wherein the Aspergillus host cell is an Aspergillus niger or Aspergillus oryzae host cell.

9. The method of claim 6, wherein the defensin is an alpha defensin, beta defensin, theta defensin, arthropod defensin, insect defensin or plant defensin.

10. The method of claims 6, which results in production of the defensin in an amount of at least 150% of the amount obtained when using a nucleic acid construct without an intron sequence.
